(19) United States
(12) Patent Application Publication
(10) Pub. No.: US 2010/0216834 A1
(43) Pub. Date: Aug. 26, 2010

(54) HIV INTEGRASE INHIBITORS

(76) Inventors: Richard C. A. Isaacs, Harleysville, PA (US); John S. Wai, Harleysville, PA (US); Linda S. Payne, Lansdale, PA (US); Thorsten E. Fisher, Hatfield, PA (US)

Correspondence Address: MERCK, P O BOX 2000, RAHWAY, NJ, 07065-0907, US

(21) Appl. No.: 12/445,594

(22) PCT Filed: Oct. 15, 2007
(86) PCT No.: PCT/US07/21982

§ 371 Date: Apr. 15, 2009

Related U.S. Application Data
(60) Provisional application No. 60/852,524, filed on Oct. 18, 2006.

(51) Int. Cl. A61K 31/4375; C07D 471/14; C07D 471/04; A61P 31/18

(52) U.S. Cl. 514/293; 540/460; 546/123

(57) ABSTRACT

Stereoisomers of compounds of Formula I are disclosed: wherein V¹, V², R^{5a}, R^{5b}, R^{5c}, R⁸ and R^{9b} are defined herein and wherein the stereoisomer contains 2 chiral centers in the 8-membered ring and one of the chiral centers is due to the presence of a chiral ring carbon. The isomers are inhibitors of HIV integrase and inhibitors of HIV replication, and are useful for the prophylaxis or treatment of infection by HIV and the prophylaxis, treatment, or delay in the onset or progression of AIDS. The compounds are employed against HIV infection and AIDS as compounds per se or in the form of pharmaceutically acceptable salts. The compounds and their salts can be employed as ingredients in pharmaceutical compositions, optionally in combination with other antivirals, immunomodulators, antibiotics or vaccines.

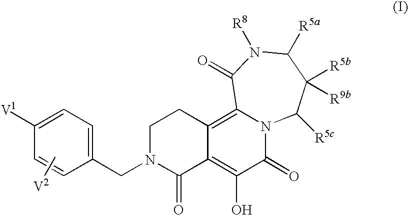

### FIELD OF THE INVENTION

The present invention is directed to certain hexahydro-diazocinonaphthyridine trione compounds and pharmaceutically acceptable salts thereof, their synthesis, and their use as inhibitors of the HIV integrase enzyme. The compounds and pharmaceutically acceptable salts thereof of the present invention are useful for preventing or treating infection by HIV and for preventing or treating or delaying the onset or progression of AIDS.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277 (1985)]. Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells. The particular advantage of the present invention is highly specific inhibition of HIV integrase and HIV replication.

The following references are of interest as background:

U.S. Pat. No. 6,380,249, U.S. Pat. No. 6,306,891, and U.S. Pat. No. 6,262,055 disclose 2,4-dioxobutyric acids and acid esters useful as HIV integrase inhibitors.

WO 01/00578 discloses 1-(aromatic- or heteroaromatic-substituted)-3-(heteroaromatic substituted)-1,3-propanediones useful as HIV integrase inhibitors.

US 2003/0055071 (corresponding to WO 02/30930), WO 02/30426, and WO 02/55079 each disclose certain 8-hydroxy-1,6-naphthyridine-7-carboxamides as HIV integrase inhibitors.

WO 02/036734 discloses certain aza- and polyaza-naphthalenyl ketones to be HIV integrase inhibitors.

WO 03/016275 discloses certain compounds having integrase inhibitory activity.

WO 03/35076 discloses certain 5,6-dihydroxypyrimidine-4-carboxamides as HIV integrase inhibitors, and WO 03/35077 discloses certain N-substituted 5-hydroxy-6-oxo-1,6-dihydropyrimidine-4-carboxamides as HIV integrase inhibitors.

WO 03/062204 discloses certain hydroxynaphthyridinone carboxamides that are useful as HIV integrase inhibitors.

WO 04/004657 discloses certain hydroxypyrrole derivatives that are HIV integrase inhibitors.

WO 2005/016927 discloses certain nitrogenous condensed ring compounds that are HIV integrase inhibitors.

### SUMMARY OF THE INVENTION

The present invention is directed to certain hydroxy-substituted 3,4,5,6,12,13-hexahydro-2H[1,4]diazocino[2,1-a]-2,6-naphthyridine-1,8,10(11H)-trione compounds. These compounds are useful in the inhibition of HIV integrase, the prevention of infection by HIV, the treatment of infection by HIV and in the prevention, treatment, and delay in the onset or progression of AIDS and/or ARC, either as compounds or their pharmaceutically acceptable salts or hydrates (when appropriate), or as pharmaceutical composition ingredients, whether or not in combination with other HIV/AIDS antivirals, anti-infectives, immunomodulators, antibiotics or vaccines. More particularly, the present invention includes individual stereoisomers of compounds of Formula I having two sources of chirality in the 8-membered ring, and pharmaceutically acceptable salts thereof:

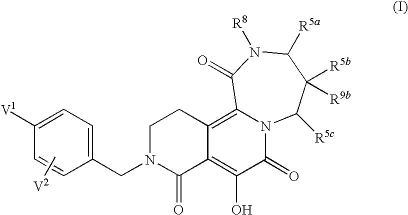

wherein:

R^{5a} is H or OH;

R^{5b} and R^{9b} are either both H or both CH₃;

R^{5c} is H or CH₃;

R⁸ is C₁₋₃ alkyl; and

V¹ and V² are each independently Br, Cl, F, or I;

and provided that

(A) when R^{5b} and R^{9b} are both H, then R^{5a} is H and R^{5c} is CH₃; and

(B) when R^{5b} and R^{9b} are both CH₃, then R^{5a} is OH and R^{5c} is H.

The foregoing provisos operate to require the presence of a chiral carbon in the 8-membered ring of the stereoisomeric compound of Formula I; i.e., proviso A renders the ring carbon to which R^{5c} is attached chiral and proviso B renders the ring carbon to which R^{5a} is attached chiral.

The present invention also includes pharmaceutical compositions containing a stereoisomer of a compound of Formula I or a pharmaceutically acceptable salt thereof. The present invention further includes methods for the treatment of AIDS, the delay in the onset or progression of AIDS, the prophylaxis of AIDS, the prophylaxis of infection by HIV, and the treatment of infection by HIV.

Other embodiments and aspects of the present invention are either further described in or will be apparent from the ensuing description, examples and appended claims.

### DETAILED DESCRIPTION OF THE INVENTION

The present invention includes individual stereoisomers of compounds of Formula I above, and pharmaceutically acceptable salts thereof. These isomers and their pharmaceutically acceptable salts are HIV integrase inhibitors (e.g., HIV-1 integrase inhibitors).

A first embodiment of the present invention (alternatively referred to herein as “Embodiment E1”) is a stereoisomer of a compound of Formula I (alternatively referred to more simply as “Stereoisomer I”), or a pharmaceutically acceptable salt thereof, wherein R⁸ is CH₃; and all other variables are as originally defined (i.e., as defined in the Summary of the Invention). In this embodiment and all subsequent embodiments, unless expressly stated to the contrary, the provisos as originally set forth in the definition of Stereoisomer I in the Summary of Invention apply.

A second embodiment of the present invention (Embodiment E2) is Stereoisomer I, or a pharmaceutically acceptable salt thereof, wherein V¹ is F; V² is Br, Cl or F; and all other variables are as originally defined or as defined in Embodiment E1.

A third embodiment of the present invention (Embodiment E3) is Stereoisomer I, or a pharmaceutically acceptable salt thereof, wherein V¹ is F; V² is Cl; and all other variables are as originally defined or as defined in Embodiment E1.

A fourth embodiment of the present invention (Embodiment E4) is Stereoisomer I, or a pharmaceutically acceptable salt thereof, wherein V¹ is F; V² is Cl and is in the meta position in the benzyl moiety; and all other variables are as originally defined or as defined in Embodiment E1. The benzyl moiety in Embodiment E3 can be represented as follows:

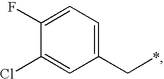

wherein the asterisk * denotes the point of attachment of the 3-chloro-4-fluorobenzyl moiety to the rest of the compound.

A fifth embodiment of the present invention (Embodiment E5) is Stereoisomer I, or a pharmaceutically acceptable salt thereof, wherein one of the sources of chirality is atropisomerism; and all other variables are as originally defined or as defined in any one of Embodiments E1 to E4.

A sixth embodiment of the present invention (Embodiment E6) is Stereoisomer I, wherein Stereoisomer I is selected from the group consisting of
Isomer A-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione (alternatively referred to herein simply as “Isomer A-1”);
Isomer B-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione (alternatively referred to herein simply as “Isomer B-1”);
Isomer A of (4S)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione (alternatively referred to herein simply as “Isomer A-2”);
Isomer B of (4S)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione (alternatively referred to herein simply as “Isomer B-2”);
Diastereomer A of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione (alternatively referred to herein simply as “Isomer A-3”);
Diastereomer B of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione (alternatively referred to herein simply as “Isomer B-3”);
Diastereomer C of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione (alternatively referred to herein simply as “Isomer C-3”);
Diastereomer D of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione (alternatively referred to herein simply as “Isomer D-3”);

and pharmaceutically acceptable salts thereof.

A seventh embodiment of the present invention (Embodiment E7) is Stereoisomer I, wherein Stereoisomer I is selected from the group consisting of
Isomer A-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione;
Diastereomer B-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione;

and pharmaceutically acceptable salts thereof.

An eighth embodiment of the present invention (Embodiment E8) is Stereoisomer I, wherein Stereoisomer I is Isomer A-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione, or a pharmaceutically acceptable salt thereof.

A ninth embodiment of the present invention (Embodiment E9) is Stereoisomer I, wherein Stereoisomer I is Diastereomer B-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione, or a pharmaceutically acceptable salt thereof.

A tenth embodiment of the present invention (Embodiment E10) is Stereoisomer I, or a pharmaceutically acceptable salt thereof, as originally defined or as defined in any of the foregoing embodiments, wherein the stereoisomer or its salt is substantially pure. As used herein “substantially pure” means that the compound or its salt is present (e.g., in a product isolated from a chemical reaction or a metabolic process) in an amount of at least about 90 wt. % (e.g., from about 95 wt. % to 100 wt. %), preferably at least about 95 wt. % (e.g., from about 98 wt. % to 100 wt. %), more preferably at least about 99 wt. %, and most preferably 100 wt. %. The level of purity of the compounds and salts can be determined using a standard method of analysis. A compound or salt of 100% purity can alternatively be described as one which is free of detectable impurities as determined by one or more standard methods of analysis.

Other embodiments of the present invention include the following:

(a) A pharmaceutical composition comprising an effective amount of Stereoisomer I and a pharmaceutically acceptable carrier.

(b) A pharmaceutical composition which comprises the product prepared by combining (e.g., mixing) an effective amount of Stereoisomer I and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, non-nucleoside HIV reverse transcriptase inhibitors, nucleoside HIV reverse transcriptase inhibitors, and HIV fusion inhibitors.

(e) A pharmaceutical combination which is (i) Stereoisomer I and (ii) an anti-HIV agent selected from the group consisting of HIV antiviral agents, immunomodulators, and anti-infective agents; wherein the compound of Formula I and the anti-HIV agent are each employed in an amount that renders the combination effective for the inhibition of HIV integrase, for the treatment or prophylaxis of infection by HIV, or for the treatment, prophylaxis or delay in the onset or progression of AIDS.

(f) The combination of (e), wherein the anti-HIV agent is an antiviral selected from the group consisting of HIV protease inhibitors, non-nucleoside HIV reverse transcriptase inhibitors, nucleoside HIV reverse transcriptase inhibitors, and HIV fusion inhibitors.

(g) A method of inhibiting HIV integrase in a subject in need thereof which comprises administering to the subject an effective amount of Stereoisomer I.

(h) A method for the treatment or prophylaxis of infection by HIV in a subject in need thereof which comprises administering to the subject an effective amount of Stereoisomer I.

(i) The method of (h), wherein Stereoisomer I is administered in combination with an effective amount of at least one antiviral selected from the group consisting of HIV protease inhibitors, non-nucleoside HIV reverse transcriptase inhibitors, nucleoside HIV reverse transcriptase inhibitors, and HIV fusion inhibitors

(j) A method for the treatment, prophylaxis, or delay in the onset or progression of AIDS in a subject in need thereof which comprises administering to the subject an effective amount of Stereoisomer I.

(k) The method of (j), wherein the compound is administered in combination with an effective amount of at least one antiviral selected from the group consisting of HIV protease inhibitors, non-nucleoside HIV reverse transcriptase inhibitors, nucleoside HIV reverse transcriptase inhibitors, and HIV fusion inhibitors

(l) A method of inhibiting HIV integrase in a subject in need thereof which comprises administering to the subject the pharmaceutical composition of (a), (b), (c) or (d) or the combination of (e) or (f).

(m) A method for the treatment or prophylaxis of infection by HIV in a subject in need thereof which comprises administering to the subject the pharmaceutical composition of (a), (b), (c) or (d) or the combination of (e) or (f).

(n) A method for the treatment, prophylaxis, or delay in the onset or progression of AIDS in a subject in need thereof which comprises administering to the subject the pharmaceutical composition of (a), (b), (c) or (d) or the combination of (e) or (f).

The present invention also includes a stereoisomeric compound of the present invention (i) for use in, (ii) for use as a medicament for, or (iii) for use in the preparation of a medicament for: (a) the inhibition of HIV integrase, (b) treatment or prophylaxis of infection by HIV, or (c) treatment, prophylaxis, or delay in the onset or progression of AIDS. In these uses, the compounds of the present invention can optionally be employed in combination with one or more anti-HIV agents selected from HIV antiviral agents, anti-infective agents, and immunomodulators.

Additional embodiments of the invention include the pharmaceutical compositions, combinations and methods set forth in (a)-(n) above and the uses set forth in the preceding paragraph, wherein the individual stereoisomer of a compound of Formula I employed therein is a stereoisomer as defined in one of Embodiments E1 to E9 described above. In all of these embodiments, the compound may optionally be used in the form of a pharmaceutically acceptable salt and may optionally be substantially pure.

The present invention also includes a composition (alternatively referred to herein as “Composition AB”) comprising a mixture of Isomer A-1 or a pharmaceutically acceptable salt thereof and Isomer B-1 or a pharmaceutically acceptable salt thereof. In a first embodiment of Composition AB (Embodiment AB-E1), Isomer A-1 is the major component of the mixture with Isomer B-1; i.e., the amount of Isomer A-1 constitutes more than 50 wt. % of the mixture (based on the weight of Isomer A-1 and Isomer B-1). In a second embodiment of Composition AB (Embodiment AB-E2), Isomer A-1 constitutes at least 70 wt. % of the mixture with Isomer B-1. In a third embodiment of Composition AB (Embodiment AB-E3), Isomer A-1 constitutes at least 90 wt. % (e.g., from about 90 wt. % to about 99 wt. %) of the mixture with Isomer B-1. In a fourth embodiment of Composition AB (Embodiment AB-E4), Isomer A-1 constitutes at least 95 wt. % (e.g., from about 95 wt. % to about 99 wt. %) of the mixture with Isomer B-1. When a pharmaceutically acceptable salt of either or both isomers is employed in the mixture, it is understood that the weight percents set forth in this paragraph are based on the free form (i.e., free acid or free base) of the isomer.

In an aspect of Composition AB as originally set forth above and of each of the foregoing embodiments thereof, the mixture of Isomer A-1 and Isomer B-1 constitutes at least about 90 wt. % (e.g., from about 95 wt. % to 100 wt. %), preferably at least about 95 wt. % (e.g., from about 98 wt. % to 100 wt. %), more preferably at least about 99 wt. %, and most preferably 100 wt. % of the composition.

Additional embodiments of the present invention include pharmaceutical compositions, combinations and methods analogous to those set forth in (a)-(n) above and uses as set forth above wherein Composition AB is employed in place of Stereoisomer I.

As used herein, the term “alkyl” refers to any linear or branched chain alkyl group having a number of carbon atoms in the specified range. Thus, for example, “C₁₋₃ alkyl” (or “C₁-C₃ alkyl”) refers to n- and isopropyl, ethyl and methyl.

The symbol “*” at the end of a bond refers to the point of attachment of a functional group or other chemical moiety to the rest of the molecule of which it is a part.

Combinations of substituents and/or variables are permissible only if such combinations result in stable compounds.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As would be recognized by one of ordinary skill in the art, compounds of the present invention can exist as tautomers. All tautomeric forms of these compounds, whether isolated or in mixtures, are within the scope of the present invention.

The second source of chirality in the 8-membered ring of Stereoisomer I is due to atropisomerism. Atropisomerism is observed when the otherwise free rotation about a bond is sufficiently restricted (e.g., by the presence of a bulky substituent) to result in rotational enantiomers called atropisomers whose interconversion is sufficiently slow to allow for their separation and characterization. See, e.g., J. March, Advanced Organic Chemistry, 4th Edition, John Wiley & Sons, 1992, pp. 101-102; and Ahmed et al., Tetrahedron 1998, 13277 for further description of atropisomerism. More particularly, the compounds of the present invention as exemplified by structure A below have sufficient hindrance to rotation along the bond indicated with the arrow to permit separation of the enantiomers (using, e.g., column chromatography on a chiral stationary phase) thereby accounting for the origin of the second chirality observed in the stereoisomers of the invention.

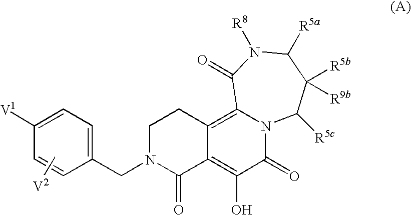

The stereoisomeric compounds of the present inventions are useful in the inhibition of HIV integrase (e.g., HIV-1 integrase), the prophylaxis or treatment of infection by HIV and the prophylaxis, treatment or the delay in the onset or progression of consequent pathological conditions such as AIDS. The prophylaxis of AIDS, treating AIDS, delaying the onset or progression of AIDS, the prophylaxis of infection by HIV, or treating infection by HIV is defined as including, but not limited to, treatment of a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by such means as blood transfusion, exchange of body fluids, bites, accidental needle stick, or exposure to patient blood during surgery.

The compounds of the present invention can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Compounds of the invention can also be employed in the form of an alkali metal salt (e.g., a sodium or potassium salt), an alkaline earth metal salt (e.g., a calcium or magnesium salt), or a salt formed with suitable organic ligands such as quaternary ammonium salts.

The term “administration” and variants thereof (e.g., “administered” or “administering”) in reference to a compound of the invention mean providing the compound to the individual in need of treatment or prophylaxis. When a compound of the invention is provided in combination with one or more other active agents (e.g., antiviral agents useful for the prophylaxis or treatment of HIV infection or AIDS), “administration” and its variants are each understood to include provision of the compound and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” (or, alternatively, “patient”) as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV integrase and thereby elicit the response being sought (i.e., an “inhibition effective amount”). When the active compound (I.e., active ingredient) is administered as the salt, references to the amount of active ingredient are to the free acid or free base form of the compound.

For the purpose of the inhibition of HIV integrase, the prophylaxis or treatment of HIV infection, or the prophylaxis or treatment or delay in the onset or progression of AIDS, the compounds of the present invention, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21^{st} edition, Lippincott Williams & Wilkins, 2005.

The compounds of this invention can be administered orally in a dosage range of about 0.001 to about 1000 mg/kg of mammal (e.g., human) body weight per day in a single dose or in divided doses. One preferred dosage range is about 0.01 to about 500 mg/kg body weight per day orally in a single dose or in divided doses. Another preferred dosage range is about 0.1 to about 100 mg/kg body weight per day orally in single or divided doses. For oral administration, the compositions can be provided in the form of tablets or capsules containing about 1.0 to about 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of the HIV integrase inhibitor compounds of the present invention with one or more anti-HIV agents useful in the treatment of HIV infection or AIDS. An “anti-HIV agent” is any agent which is directly or indirectly effective in the inhibition of HIV integrase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more HIV antivirals, immunomodulators, antiinfectives, or vaccines useful for treating HIV infection or AIDS, such as those disclosed in Table 1 of WO 01/38332 or in the Table in WO 02/30930. Suitable HIV antivirals for use in combination with the compounds of the present invention include, for example, those listed in Table A as follows:

TABLE A

| Name | Type |
|---|---|
| abacavir, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, Retrovir ® | nRTI |
| capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, Rescriptor ® | nnRTI |
| efavirenz, Sustiva ®, Stocrin ® | nnRTI |
| efavirenz + emtricitabine + tenofovir DF, | nnRTI + nRTI |
| Atripla ® | |
| emtricitabine, FTC, Emtriva ® | nRTI |
| emtricitabine + tenofovir DF, Truvada ® | nRTI |
| emvirine, Coactinon ® | nnRTI |
| enteric coated didanosine, Videx EC ® | nRTI |
| enfuvirtide, Fuzeon ® | FI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| MK-0518 (Merck) | InI |
| nelfinavir, Viracept ® | PI |
| nevirapine, Viramune ® | nnRTI |
| PPL-100 (also known as PL-462) (Ambrilia) | PI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), | nRTI |
| Viread ® | |
| tipranavir, Aptivus ® | PI |

FI = fusion inhibitor;
InI = integrase inhibitor;
PI = protease inhibitor;
nRTI = nucleoside reverse transcriptase inhibitor;
nnRTI = non-nucleoside reverse transcriptase inhibitor.
Some of the drugs listed in the table are used in a salt form; e.g., indinavir sulfate, atazanvir sulfate, nelfinvavir mesylate.

It will be understood that the scope of combinations of the compounds of this invention with HIV antivirals, immunomodulators, anti-infectives or vaccines is not limited to the foregoing substances or to the list in the above-referenced Tables in WO 01/38332 and WO 02/30930, but includes in principle any combination with any pharmaceutical composition useful for the treatment of HIV infection or AIDS. The HIV antivirals and other agents will typically be employed in these combinations in their conventional dosage ranges and regimens as reported in the art, including, for example, the dosages described in the Physicians' Desk Reference, 58^{th} edition, Thomson P D R, 2004, or the 59^{th} edition thereof, 2005. The dosage ranges for a compound of the invention in these combinations are the same as those set forth above. It is understood that pharmaceutically acceptable salts of the compounds of the invention and/or the other agents (e.g., indinavir sulfate) can be used as well.

The present invention also includes a process (Process P1) for preparing a compound of Formula II:

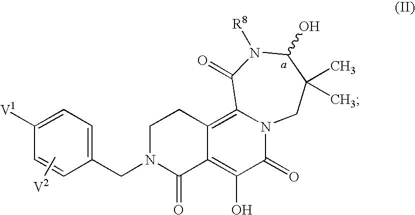

which comprises:

(A) treating a compound of Formula III:

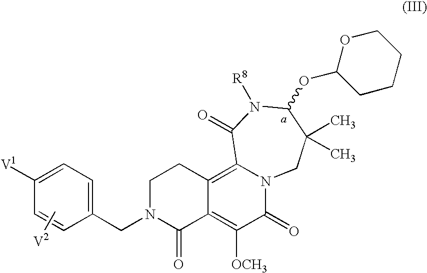

with acid to obtain Compound II; wherein stereocenter “a” is in the R or the S configuration; R⁸ is C₁₋₃ alkyl; and V¹ and V² are each independently Br, Cl, F, or I.

Example 38 of WO ______ (corresponding to International Application No. PCT/US2005/017369, filed May 5, 2006) discloses the preparation of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione via (3R)-3-(benzyloxy)-4,4-dimethyldihydrofuran-2(3H)-one which is prepared from D(−)-pantolactone. Example 39 discloses the preparation of (4S)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione in the same manner except substituting L-(+)-pantolactone for D(−)-pantolactone. It has been discovered that the preparative routes disclosed in Examples 38 and 39 do not respectively provide the 4R and 4S enantiomers, but instead provide a racemic mixture thereof. It has also been discovered that the installation of the benzyl protective group on the hydroxyl group of optically pure L and D-pantolactones employed in Examples 38 and 39 results in racemization of the chiral centers, whereas installation of a 2-tetrahydropyranyl protective group as employed in Process P1 does not. See Example 1, steps 10 et seq. below.

A first embodiment of Process P1 (Embodiment P1-E1) is Process P1 as originally defined which further comprises:

(B) sequentially treating a compound of Formula IV:

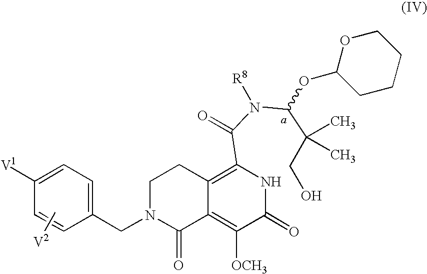

first with a sulfonic anhydride or a sulfonyl halide in the presence of a first base and then with a second base to obtain Compound III; and all other variables are as originally defined.

A second embodiment of Process P1 (Embodiment P1-E2) is Process P1 as originally defined, wherein Compound II is a compound of Formula II-A:

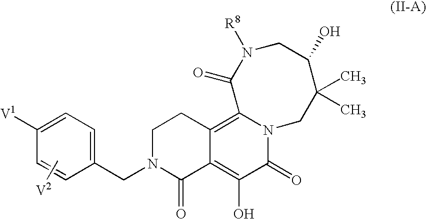

and Compound III is a compound of Formula

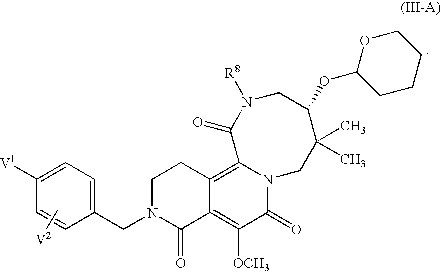

A third embodiment of Process P1 (Embodiment P1-E3) is Process P1 as defined in the second embodiment, which further comprises:

(B) sequentially treating a compound of Formula IV-A:

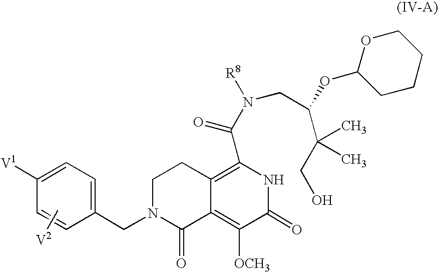

first with a sulfonic anhydride or a sulfonyl halide in the presence of a first base and then with a second base to obtain Compound III-A.

A fourth embodiment of Process P1 (Embodiment P1-E4) is Process P1 as originally defined or as defined in any one of Embodiments P1-E1 to P1-E3, wherein R⁸ is CH₃; and all other variables are as originally defined.

A fifth embodiment of Process P1 (Embodiment P1-E5) is Process P1 as originally defined or as defined in any one of the foregoing embodiments, wherein V¹ is F and V² is Cl in the meta position of the benzyl moiety.

Step A of Process P1 is a deprotection step in which the ether substituents on the ring are converted to OH groups in the presence of an acid. The acid can be either a proton acid or a Lewis acid. Suitable acids include, for example, boron halides (e.g., BBr₃ or Me₂BBr), trialkylsilyl halides (e.g., trimethylsilyl iodide), aluminum halides (e.g., aluminum chloride), and hydrogen halides (e.g., HBr).

Step A is typically conducted in a solvent. The solvent in Step A can be any organic compound which under the reaction conditions employed is in the liquid phase, is chemically inert, and will dissolve, suspend, and/or disperse the reactants so as to bring the reactants into contact and permit the reaction to proceed. When the acid employed is a boron halide, a trialkylsilyl halide, or an aluminum halide, the solvent is suitably a halohydrocarbon (e.g., methylene chloride or chloroform) or a dialkyl sulfide (e.g., dimethyl sulfide) or a combination thereof. When the acid is a hydrogen halide, the solvent is suitably an alkylcarboxylic acid (e.g., a C₁₋₄ alkylcarboxylic acid such as acetic acid).

Step A can be conducted at any temperature at which the reaction will detectably proceed. Step A can be suitably conducted at a temperature in a range of from about −78° C. to about 50° C., and is typically conducted at a temperature in a range of from about 0 to about 40° C. In one embodiment, the temperature is in a range of from about 15° C. to about 30° C. (e.g., from about 18° C. to about 25° C.).

The acid can be employed in Step A in any proportion with respect to Compound III which will result in the formation of at least some of Compound II. Typically, however, the acid is employed in an amount which can optimize the conversion of Compound III to Compound II. In one embodiment, the acid is employed in Step A in an amount of at least 1 equivalent (e.g., from about 1 to about 15 equivalents) per equivalent of Compound III. In another embodiment, the acid is employed in an amount of from about 4 to about 10 equivalents per equivalent of Compound III.

Step A can be conducted by adding acid dissolved in a solvent (e.g., BBr₃ in methylene chloride) to a cold (e.g., less than about 0° C.) solution of Compound III in the same solvent, bringing the resulting mixture to reaction temperature, and maintaining the mixture at reaction temperature until the reaction is complete or the desired degree of conversion of the reactants is achieved. The order of addition of the reactants and reagents to the reaction vessel is not critical; i.e., they can be charged concurrently or sequentially in any order. The reaction is generally conducted under an inert atmosphere (e.g., nitrogen or argon gas). The reaction time can vary widely depending upon, inter alia, the reaction temperature and the choice and relative amounts of reactants and reagents, but the reaction time is typically in the range of from about 0.5 to about 24 hours. Compound II can be separated from the reaction mixture using conventional techniques, such as diluting the reaction mixture with additional solvent and water, separating the resulting organic and aqueous phases, and then washing, drying, filtering, and concentrating the organic phase. The atropisomers of Compound II can be separated by chromatography.

Step B of Process P1 results in the formation of Compound III. Step B comprises a reaction sequence in which the OH group is first converted to a sulfonate ester by treatment of Compound IV with a sulfonic anhydride or a sulfonyl halide in the presence of a first base, and the sulfonate ester is then cyclized by treatment with a second base to provide Compound III. Suitable sulfonic anhydrides include alkanesulfonic anhydrides, haloalkanesulfonic anhydrides, and arenesulfonic anhydrides. Suitable sulfonyl halides include alkanesulfonyl halides, haloalkanesulfonyl halides, and arenesulfonyl halides. The sulfonic anhydride can be, for example, methanesulfonic anhydride, trifluoromethanesulfonic anhydride, p-toluenesulfonic anhydride, or benzenesulfonic anhydride. The sulfonyl halide can be, for example, methanesulfonyl chloride, trifluoromethanesulfonyl chloride, p-toluenesulfonyl chloride, or benzenesulfonyl chloride. The first base is suitably a tertiary amine such as TEA, DIPEA, pyridine, or 4-N,N-dimethylaminopyridine. The second base is suitably an alkali metal carbonate such as cesium carbonate, sodium carbonate, or potassium carbonate.

Step B is typically conducted in one or more solvents. The solvent(s) in Step B can be any organic compound which under the reaction conditions employed is in the liquid phase, is chemically inert, and will dissolve, suspend, and/or disperse the reactants so as to bring the reactants into contact and permit the reaction to proceed. In the sulfonation reaction, the solvent is suitably a halohydrocarbon (e.g., methylene chloride or chloroform) or pyridine. In the cyclization reaction, the solvent is suitably a tertiary amide, an ether, or a dialkylsulfoxide. The solvent can be, for example, DMF, DMA, DMSO, THF, DME, or dioxane.

Step B can be conducted at any temperature at which the reaction will measurably proceed. The sulfonation in Step B is suitably conducted at a temperature in a range of from about −78° C. to about 50° C., and is typically conducted at a temperature in a range of from about 0° C. to about 40° C. The cyclization in Step B is suitably conducted at a temperature in a range of from about 80° C. to about 160° C., and is typically conducted at a temperature in a range of from about 100° C. to about 160° C.

The sulfonic anhydride or sulfonyl halide, the first base, and the second base can be employed in Step B in any proportion with respect to Compound IV which will result in the formation of at least some of Compound III. Typically, however, they are each employed in an amount which can optimize the conversion of Compound IV to Compound III. In one embodiment, the sulfonating agent is employed in an amount of at least 2 equivalents (e.g., from about 2 to about 4 equivalents) per equivalent of Compound IV; the first base is employed in an amount of at least 2 equivalents (e.g., from about 2 to about 4 equivalents) per equivalent of Compound IV; and the second is employed is employed in an amount of at least 2 equivalents (e.g., from about 2 to about 6 equivalents) per equivalent of Compound IV.

Step B can be conducted by adding the sulfonic anhydride (or sulfonyl chloride) to a reaction vessel containing a solution of Compound IV and the first base in a solvent (e.g., a halohydrocarbon), bringing the resulting mixture to reaction temperature, and maintaining the mixture at reaction temperature until the reaction is complete or the desired degree of conversion of the reactants is achieved. The order of addition of the reactants and reagents to the reaction vessel is not critical; i.e., they can be charged concurrently or sequentially in any order. The reaction is generally conducted under an inert atmosphere (e.g., nitrogen or argon gas). The reaction time can vary widely depending upon, inter alia, the reaction temperature and the choice and relative amounts of reactants and reagents, but the reaction time is typically in the range of from about 0.5 to about 24 hours. The resulting sulfonate ester product can be subsequently recovered by, for example, diluting the product mixture with an organic solvent (e.g., chloroform), washing the diluted mixture with water, separating the organic and aqueous phases, and then drying, filtering and concentrating the organic phase. The sulfonate product can then be mixed with the second base in a solvent (e.g., anhydrous DMF), the mixture heated (e.g., in a microwave oven or via another conventional heat source such as an oil bath) to reaction temperature, and the mixture then maintained at reaction temperature until the reaction is complete or the desired degree of conversion of the reactants is achieved. The reaction time for the cyclization can vary widely depending upon the same factors as noted above in the sentence describing the sulfonation reaction time, but is typically in a range of from about 0.5 to about 12 hours. The cyclized product can then be recovered using conventional techniques.

The present invention further includes a compound which is a compound of Formula III, a compound of Formula IV, or a salt thereof. (i.e., in this context the salt is not limited to a pharmaceutically acceptable salt). In a first embodiment, the compound is a compound of Formula a compound of Formula IV-A, or a salt thereof. In a second embodiment, the compound is 6-(3-chloro-4-fluorobenzyl)-N-[(2R)-4-hydroxy-3,3-dimethyl-2-(tetrahydro-2H-pyran-2-yloxy)butyl]-4-methoxy-N-methyl-3,5-dioxo-2,3,5,6,7,8-hexahydro-2,6-naphthyridine-1-carboxamide; (4R)-11-(3-chloro-4-fluorobenzyl)-9-methoxy-2,5,5-trimethyl-4-(tetrahydro-2H-pyran-2-yloxy)-3,4,5,6,12,13-hexahydro-2H[1,4]diazocino[2,1-a]-2,6-naphthyridine-1,8,10(11H)-trione; or a salt thereof.

Abbreviations employed herein include the following: Bu=butyl; DIPEA=diisopropylethylamine; DMA=N,N-dimethylacetamide; DME=1,2-dimethoxyethane; DMF=N,N-dimethylformamide; DMSO=dimethylsulfoxide; EDC or EDAC=1-ethyl-3-(3-dimethylaminopropyl) carbodiimide; ES MS=electrospray mass spectroscopy; Et=ethyl; EtOAc=ethyl acetate; FBS=fetal bovine serum; HOAT=1-hydroxy-7-azabenzotriazole; HPLC=high performance liquid chromatography; Me=methyl; MeOH=methanol; MTBE=methyl tert-butyl ether; NMR=nuclear magnetic resonance; TEA=triethylamine; TFA=trifluoroacetic acid; THF=tetrahydrofuran.

The following examples serve only to illustrate the invention and its practice. The examples are not to be construed as limitations on the scope or spirit of the invention.

### Example 1

Isomers A-1 and B-1 of (4R)-11-(3-Chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione

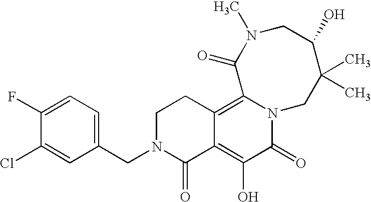

### Step 1: 1-(3-Chloro-4-fluorobenzyl)piperidin-2-one

To a cold (0° C.) solution of valerolactam (153.30 g, 1.54 mol) in mixture of anhydrous 1-methyl-2-pyrrolidinone (3.5 L) and THF (350 mL), sodium hydride (67.7 g, 1.69 mol, 60% dispersion in oil) was added over a period of 5 minutes. The reaction mixture was stirred for 30 minutes, and a solution of 3-chloro-4-fluorobenzylbromide (345.5 g, 1.54 mol) in 1-methyl-2-pyrrolidinone (200 mL) was added over 30 minutes at 0° C. The reaction mixture was stirred at 0° C. for 1 hour, and was allowed to warm up and stirred at room temperature overnight. The reaction mixture was quenched with distilled water (5 L), and extracted with dichloromethane (three times; 2 L, 1 L, 1 L). The organic extracts were combined, washed with water (3×; 4 L each time). The residual oil was dissolved in ethyl acetate (4 L), and extracted with water (3×; 2 L each time). The organic layer was separated, concentrated under vacuum to give the title product that solidified upon standing.

¹H NMR (400 MHz, CDCl₃) δ 7.24 (m, 2H), 7.0 (m, 2H), 7.1 (m, 1H), 4.56 (s, 2H), 3.19 (t, J=4.9 Hz, 2H), 2.46 (t, J=6.4 Hz, 211), 1.8-1.75 (m, 41-1).

### Step 2: 1-(3-Chloro-4-fluorobenzyl)-5,6-dihydropyridin-2(1H)-one

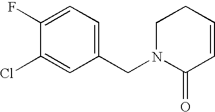

To a cold (−20° C.) solution of 1-(3-chloro-4-fluorobenzyl)piperidin-2-one (340 g, 1.41 mol) in anhydrous tetrahydrofuran (5 L) under an atmosphere of nitrogen, a solution of lithium bis(trimethylsilyl)amide (3.09 L, 3.09 mol; 1M in THF) was added over a period of 40 minutes with the temperature of the reaction maintained at −20° C. After the addition was complete, the reaction mixture was stirred at −20° C. for one hour. Methyl benzene sulfonate (231 mL, 1.69 mol) was added to the reaction mixture over a period of 30 minutes. The reaction mixture was stirred at −20° C. for 30 minutes. The product mixture was diluted with ethyl acetate (4 L) and washed with water (four times; 2 L each time). The organic extract was concentrated under vacuum. The residue was dissolved in toluene (4 L), treated with solid sodium carbonate (500 g), and heated at 100° C. for one hour. The product mixture was diluted with ethyl acetate (4 L) and washed with water (4 times; 2 L each). The organic extract was concentrated under vacuum. The residue was subjected to column chromatography on silica gel eluting with a gradient of 0-60% EtOAc in heptane. Collection and concentration of appropriate fractions provide the title compound as oil.

¹H NMR (400 MHz, CDCl₃) δ 7.3 (m, 1H), 7.15 (m, 1H), 7.1 (t, 1H), 6.6 (m, 1H), 6.0 (m, 1H), 4.55 (s, 2H), 3.33 (t, 2H), 1.38 (m, 21-1). ES MS M+1=240.13

### Step 3: 2-Butoxy-2-oxoethanaminium chloride

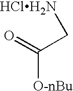

To a suspension of glycine hydrochloride (400 g, 3.58 mol) in n-butanol (8 L), thionyl chloride (1.37 L, 18.84 mol) was added slowly dropwise. After addition was complete, the reaction was heated at 70° C. overnight. The product mixture was concentrated under vacuum and the residue was triturated with a mixture of heptane/ethyl acetate. The white solid precipitated was filtered and dried under a stream of dry nitrogen to provide the title compound.

¹H NMR (400 MHz, CDCl₃) δ 8.5 (br s, 3H), 4.18 (t, J=6.7 Hz, 2H), 4.0 (br s, 2H), 1.62 (m, 2H), 1.38 (m, 2H), 0.92 (t, J=7.4 Hz, 3H). ES MS M+1=132.

### Step 4: Butyl N-[ethoxy(oxo)acetyl]glycinate

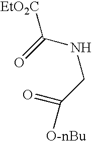

A mixture of 2-butoxy-2-oxoethanaminium chloride (573.5 g, 3.42 mol), triethylamine (415 g, 4.1 mol), and diethyl oxalate (1.0 kg, 6.8 mol) in ethanol (7 L) was heated at 50° C. for 3 hours. The product mixture was cooled and concentrated under vacuum. The residue was dissolved in methylene chloride and washed with two 4 L portions of water. The organic fraction was dried over anhydrous magnesium sulfate, filtered, and concentrated under vacuum. The residual oil was subjected to column chromatography on silica gel eluting with heptane/ethyl acetate gradient. Collection and concentration of appropriate fractions provided the title material.

¹H NMR (400 MHz, CDCl₃) δ 7.56 (br s, 1H), 4.37 (q, J=7.2 Hz, 2H), 4.2 (t, J=6.6 Hz, 2H), 4.12 (d, J=5.5 Hz, 2H), 1.64 (p, J=6.8 Hz, 2H), 1.39 (t, J=7.15 Hz, 3H), 1.37 (m, 2H), 0.94 (t, J=7.4 Hz, 3H). ES MS M+1=232.

Alternative route. The glycinate was also prepared using ethyl oxalyl chloride in place of diethyl oxalate as follows: To a mixture of 2-butoxy-2-oxoethanaminium chloride (1.48 Kg, 8.85 mol), dichloromethane (10.6 L), and deionized water (10.6 L) at room temperature, potassium bicarbonate (2.2 Kg, 22.1 mol) was added in three portions. The endothermic mixture was warmed back to 16° C. Ethyl oxalyl chloride (1.08 L, 9.74 mol) was added via an addition funnel over 45 minutes, and stirred at room temperature for two hours. The aqueous layer was separated and extracted with dichloromethane (2×2 L). The organic fractions were combined, and washed with a mixture of deionized water (10 L) and brine (1.5 L). The organic fraction was concentrated under vacuum to provide the title material.

¹H NMR (400 MHz, CDCl₃) δ 7.56 (br s, 1H), 4.37 (q, J=7.2 Hz, 2H), 4.2 (t, J=6.6 Hz, 2H), 4.12 (d; J=5.5 Hz, 2H), 1.64 (p, J=6.8 Hz, 2H), 1.39 (t, J=7.15 Hz, 3H), 1.37 (m, 2H), 0.94 (t, J=7.4 Hz, 3H). ES MS M+1=232.

### Step 5: Ethyl 5-butoxy-1,3-oxazole-2-carboxylate

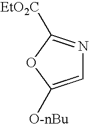

To a solution of butyl N-[ethoxy(oxo)acetyl]glycinate (783 g, 3.38 mol) in acetonitrile (8 L) in a 50 L glass reactor with overhead stirrer, phosphorus pentoxide (415 g, 2.92 mol) was added in portions. The reaction was heated at 60° C. for 1 hour. The product mixture was cooled, and water (8 L) was added with the mixture maintained at 20° C. The resultant mixture was extracted with dichloromethane (8 L, and 3 times 2 L). The organic extracts were combined, washed twice with saturated aqueous sodium bicarbonate (8 L total), dried over anhydrous magnesium sulfate, filtered and concentrated under vacuum. The residual oil was subjected to column chromatography on silica gel eluting with 0-30% heptane/ethyl acetate gradient. Collection and concentration of appropriate fractions provided the title material.

¹H NMR (400 MHz, CDCl₃) δ 6.33 (s, 1H), 4.42 (q, J=7.2 Hz, 2H), 4.18 (t, J=6.4 Hz, 2H), 1.8 (p, J=6.4 Hz, 2H), 1.47 (p, J=7.4 Hz, 2H), 1.41 (t, J=7.15 Hz, 3H), 0.97 (t, J=7.4 Hz, 3H). ES MS M+1=214.

### Step 6: Ethyl 6-(3-chloro-4-fluorobenzyl)-4-hydroxy-5-oxo-5,6,7,8-tetrahydro-2,6-naphthyridine-1-carboxylate

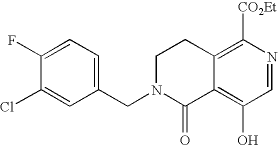

A mixture of ethyl 5-butoxy-1,3-oxazole-2-carboxylate (248 g, 1.16 mol; step 5), 1-(3-chloro-4-fluorobenzyl)-5,6-dihydropyridin-2(1H)-one (199.2 g, 0.83 mol; step 2), and deionized water (22.5 mL, 1.25 mol) in a glass liner of a stainless steel high pressure reactor (with the interstitial space between the liner and the pressure vessel was filled with water) was heated at 135° C. with stirring for 72 hours. The product mixture was cooled in an ice-water bath and the gaseous by-product was carefully vented. The orange solid product was triturated with methyl tert-butyl ether (300 mL) and collected by filtration. The product recrystallized from boiling ethanol-water (˜500 mL, 9:1 v/v), collected by filtration, washed successively with a small quantity of ethanol, methyl tert-butyl ether (300 mL), and heptane (200 mL), and air dried to afford the title compound.

¹H NMR (400 MHz, CDCl₃) δ 12.79 (s, 1H), 8.42 (s, 1H), 7.4 (dd, J=2, 7 Hz, 1H), 7.2 (m, 1H), 7.15 (t, J=8.6 Hz, 1H), 4.7 (s, 2H), 4.4 (q, J=7 Hz, 2H), 3.5 (m, 4H), 1.4 (t, J=7 Hz, 3H). (ES MS M+1=379.0)

### Step 7: Ethyl 6-(3-chloro-4-fluorobenzyl)-4-methoxy-5-oxo-5,6,7,8-tetrahydro-2,6-naphthyridine-1-carboxylate

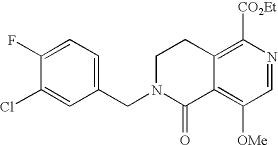

To a stirred solution of ethyl 6-(3-chloro-4-fluorobenzyl)-4-hydroxy-5-oxo-5,6,7,8-tetrahydro-2,6-naphthyridine-1-carboxylate (208 g, 0.55 mol) in a mixture of dichloromethane (830 mL) and methanol (410 mL) at 10° C., a solution of (trimethyl-silyl)diazomethane (600 mL, 1.2 mol; 2M) in hexanes was added over a period of 1 hour with the reaction temperature maintained below 15° C. The reaction mixture (unstirred) was allowed to stand at 10° C. overnight, and then at 20° C. for additional 4 hours. The reaction mixture was cooled back to 10° C. and quenched with acetic acid (˜75 mL). The product mixture was concentrated under vacuum and the residue recrystallized from boiling methyl tert-butyl ether and heptane. The solid recrystallized was collected by filtration, washed with a mixture of methyl tert-butyl ether and heptane (1:1, v/v), and air dried to afford the title compound.

¹H NMR (400 MHz, CDCl₃) δ 8.42 (s, 1H), 7.41 (dd, J=2, 7 Hz, 1H), 7.24 (m, 1H), 7.11 (t, J=8.6 Hz, 1H), 4.70 (s, 2H), 4.42 (q, J=7 Hz, 2H), 4.12 (s, 3H), 3.4 (m, 4H), 1.42 (t, J=7 Hz, 3H). (ES MS M+1=392.9)

### Step 8: Ethyl 3-(acetyloxy)-6-(3-chloro-4-fluorobenzyl)-4-methoxy-5-oxo-5,6,7,8-tetrahydro-2,6-naphthyridine-1-carboxylate

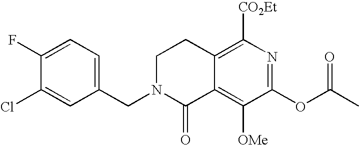

To a cold (5° C.) mixture of ethyl 6-(3-chloro-4-fluorobenzyl)-4-methoxy-5-oxo-5,6,7,8-tetrahydro-2,6-naphthyridine-1-carboxylate (199 g, 0.51 mol) and urea hydrogen peroxide (100 g, 1.06 mol) in dichloromethane (1.5 L), trifluoroacetic anhydride was added dropwise over a period of 45 minutes. The resultant homogeneous solution was stirred at 20° C. for 30 minutes and cooled back to 5° C. The reaction mixture was treated with aqueous potassium hydrogen phosphate (pH of aqueous extract increased to ˜8), followed by slow addition of freshly prepared aqueous sodium bisulfite solution with the temperature of the product mixture maintained below 25° C. The organic extract was separated and the aqueous fraction extracted with toluene (2×). The organic extracts were combined, dried over anhydrous sodium sulfate, filtered, and concentrated under vacuum. Without further purification, a solution of this intermediate N-oxide (˜280 g) and acetic anhydride (239 mL, 2.5 mol) in toluene (2 L) was heated at 110° C. for 16 hours. The product mixture was concentrated under vacuum. The resultant oil was concentrated from toluene (300 mL, twice) and stored under vacuum overnight. The acetate product was used in the following step without further purification.

(ES MS M+1=408.9)

### Step 9: 6-(3-Chloro-4-fluorobenzyl)-4-methoxy-3,5-dioxo-2,3,5,6,7,8-hexahydro-2,6-naphthyridine-1-carboxylic acid

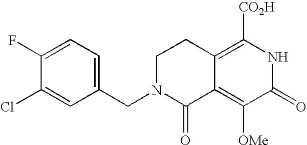

A mixture of ethyl 3-(acetyloxy)-6-(3-chloro-4-fluorobenzyl)-4-methoxy-5-oxo-5,6,7,8-tetrahydro-2,6-naphthyridine-1-carboxylate (217 g, 0.48 mol), lithium hydroxide monohydrate (70.7 g, 1.67 mol), and water (320 mL) in ethanol (1.8 L) was sonicated for 20 minutes. The reaction mixture was cooled in an ice-water bath and treated with hydrochloric acid (425 mL, 3 M). The resultant light yellow solid was filtered, washed successively with water (1 L), a 3:2 v/v mixture of water and ethanol (500 mL), MTBE (750 mL), and air dried. The yellow solid was dissolved in anhydrous DMF (700 mL) and concentrated under vacuum. The procedure was repeated twice to remove residual water. The yellow solid was triturated with MTBE, filtered, and stored under vacuum overnight to afford the title acid.

¹H NMR (400 MHz, CDCl₃) δ 7.54 (dd, J=2, 7 Hz, 1H), 7.3 (m, 2H), 4.65 (s, 2H), 3.89 (s, 3H), 3.43 (t, J=5.5 Hz, 2H), 3.00 (t, J=5.5 Hz, 2H). (ES MS M+1=380.9)

### Step 10: (3R)-4,4-Dimethyl-3-(tetrahydro-2H-pyran-2-yloxy)dihydrofuran-2(3H)-one

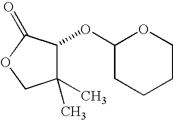

To a mixture of D(−)-pantolactone (10.0 g, 76.8 mmol) and p-toluenesulfonic acid monohydrate (0.1 g, 0.5 mmol) in anhydrous methylene chloride (130 mL) under an atmosphere of nitrogen at room temperature, 3,4-dihydro-2H-pyran was added dropwise over a period of 20 minutes. (See Ito et al., Synthesis 1993, pp 137-140; Szabo et al., Tetrahedron Asymmetry 1999, 10: pp 61-76). The reaction mixture was stirred at the same temperature for 45 minutes. The product mixture was treated with water (150 mL) and diluted with dichloromethane (150 mL). The organic extract was washed with brine, dried over anhydrous magnesium sulfate, filtered, and concentrated under vacuum. The residue was subjected to purification on silica gel eluting with 0-40% ethyl acetate in hexane gradient. Collection and concentration of appropriate fractions provided the title compound as a mixture of diastereoisomers.

¹H NMR (400 MHz, CDCl₃) δ 5.16 (t, J=3.7 Hz, 0.73H), 4.86 (t, J=2.9 Hz, 0.27H), 5.24-3.53 (m), 1.22 (s, 2.2H), 1.20 (s, 0.8H), 1.14 (s, 2.2H), 1.11 (s, 0.8H).

### Step 11: (2R)-4-Hydroxy-N,3,3-trimethyl-2-(tetrahydro-2H-pyran-2-yloxy)-butanamide

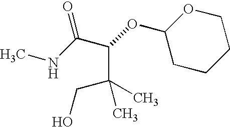

To a cold (0° C.) solution of methylamine in methanol (7.6 mL; 40% aqueous solution) in methanol (70 mL), (3R)-4,4-Dimethyl-3-(tetrahydro-2H-pyran-2-yloxy)dihydrofuran-2(3H)-one (15 g, 70 mmol) was added. The reaction mixture was stirred at the room temperature for 3 hours. The product mixture was concentrated under vacuum. The residue was subjected to purification on silica gel eluting with 20-100% ethyl acetate in hexane gradient. Collection and concentration of appropriate fractions provided the title compound as a mixture of diastereoisomers.

¹H NMR (400 MHz, CDCl₃) δ 6.76 (br signal, 0.27H), 6.35 (br signal, 0.73H), 4.38-3.18 (m), 2.86 (d, J=5.1 Hz, 2.2H), 2.85 (d, J=5.6 Hz, 0.8H), 1.03 (s, 3H), 0.88 (s, 3H).

### Step 12: (3R)-2,3-Dimethyl-4-(methylamino)-3-(tetrahydro-2H-pyran-2-yloxy)-butan-1-ol

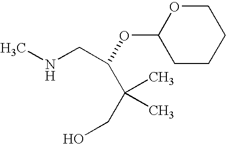

A solution of (2R)-4-Hydroxy-N,3,3-trimethyl-2-(tetrahydro-2H-pyran-2-yloxy)-butanamide (11.6 g, 47.3 mmol) in anhydrous THF (90 mL) under an atmosphere of nitrogen was treated with a solution of lithium aluminum hydride in THF (142 mL, 1M, 142 mmol). The reaction mixture was heated in an oil bath at 77° C. for 72 hours. The product mixture was cooled with an ice-water bath and was treated successively with water (5.4 mL), 15% aqueous NaOH (5.4 mL), and water (16.2 mL). The resultant slurry was stirred at room temperature for 1 hour and filtered through a pad of Celite. The solid was washed with THF. The combined filtrate was dried over anhydrous sodium sulfate, filtered, and concentrated under vacuum. The residue was concentrated from benzene under vacuum to afford the title compound as a mixture of diastereoisomers.

¹H NMR (400 MHz, CDCl₃) δ 4.68 (m, 0.73H), 4.47 (m, 0.27H), 3.9-2.4 (m), 2.46 (s, 0.8H), 2.43 (s, 2.2H), 0.97 (s, 0.8H), 0.95 (s, 2.2H), 0.93 (s, 2.2H), 0.85 (s, 0.8H).

ES-MS M+1=232.

### Step 13: 6-(3-Chloro-4-fluorobenzyl)-N-[(2R)-4-hydroxy-3,3-dimethyl-2-(tetrahydro-2H-pyran-2-yloxy)butyl]-4-methoxy-N-methyl-3,5-dioxo-2,3,5,6,7,8-hexahydro-2,6-naphthyridine-1-carboxamide

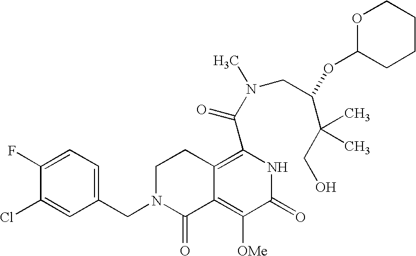

A mixture of 6-(3-chloro-4-fluorobenzyl)-4-methoxy-3,5-dioxo-2,3,5,6,7,8-hexahydro-2,6-naphthyridine-1-carboxylic acid (15.8 g, 41.5 mmol), (3R)-2,3-dimethyl-4-(methylamino)-3-(tetrahydro-2H-pyran-2-yloxy)-butan-1-ol (9.6 g, 41.5 mmol), EDC (9.6 g, 49.8 mmol), HOAt (0.28 g, 2.1 mmol) and N-methylmorpholine (22.9 mL, 207 mmol) in anhydrous methylene chloride (300 mL) was stirred at room temperature overnight. The product solution was diluted with methylene chloride and washed successively with water and brine. The organic extract was dried over anhydrous sodium sulfate, filtered, and concentrated under vacuum. The residue was subjected to column chromatography on silica gel eluting with 0-10% methanol/chloroform gradient. Collection and concentration of appropriate fractions provided the title material as a mixture of two diastereoisomers. ES-MS M+H=594 for both isomers.

### Step 14: (4R)-11-(3-Chloro-4-fluorobenzyl)-9-methoxy-2,5,5-trimethyl-4-(tetrahydro-2H-pyran-2-yloxy)-3,4,5,6,12,13-hexahydro-2H[1,4]diazocino[2,1-a]-2,6-naphthyridine-1,8,10(11H)-trione

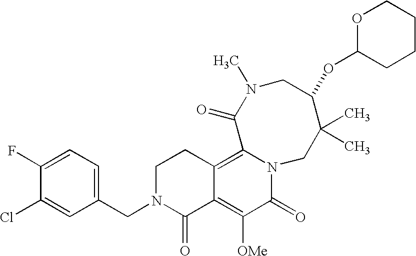

To a solution of 6-(3-chloro-4-fluorobenzyl)-N-[(2R)-4-hydroxy-3,3-dimethyl-2-(tetrahydro-2H-pyran-2-yloxy)butyl]-4-methoxy-N-methyl-3,5-dioxo-2,3,5,6,7,8-hexahydro-2,6-naphthyridine-1-carboxamide (4.0 g, 6.7 mmol) and diisopropylethylamine (2.6 mL, 14.8 mmol) in dichloromethane (34 mL) at room temperature, methanesulfonic anhydride (2.3 g, 13.5 mmol) was added. The reaction mixture was stirred at room temperature for 1 hour. The product mixture was diluted with methylene chloride and washed with water. The organic extract was dried over anhydrous sodium sulfate, filtered, and concentrated under vacuum. This intermediate mixture of mono- and bis-mesylates was used in the following cyclization reaction without further purification.

A mixture of the above mesylates (0.88 g, 1.17 mmol) and cesium carbonate (1.52 g, 4.69 mmol) in anhydrous DMF (18 mL) was heated in a microwave oven at 150° C. for 30 minutes. The reaction mixture was filtered through a pad of Celite and the solid filtered washed with DMF. Filtrates from five consecutive runs were combined and concentrated under vacuum. The residue was partitioned between ethyl acetate and brine. The organic extract was washed with brine, dried over anhydrous sodium sulfate, filtered and concentrated under vacuum. The residue was subjected to column chromatography on silica gel eluting with 0-5% methanol/chloroform gradient. Collection and concentration of appropriate fractions afforded the title compound as a mixture of two diastereoisomers.

¹H NMR (400 MHz, CDCl₃) δ 7.37 (dd, J=1.8, 6.8 Hz, 1H), 7.21 (m, 1H), 7.10 (t, J=8.6 Hz, 1H), 4.86-4.49 (m), 4.08 (s), 4.07 (s), 4.2-2.8 (m), 3.17 (s), 3.13 (s), 1.8-1.4 (br m), 1.25 (s), 1.10 (s), 0.95 (s), 0.91 (s). ES-MS M+H=576 for both isomers.

Alternative route. The title intermediate was also prepared as follows: To a solution of 6-(3-chloro-4-fluorobenzyl)-N-[(2R)-4-hydroxy-3,3-dimethyl-2-(tetrahydro-2H-pyran-2-yloxy)butyl]-4-methoxy-N-methyl-3,5-dioxo-2,3,5,6,7,8-hexahydro-2,6-naphthyridine-1-carboxamide (20.0 g, 33.7 mmol) and diisopropylethylamine (12.9 mL, 74.1 mmol) in dichloromethane (168 mL) at room temperature, methanesulfonic anhydride (12.3 g, 70.7 mmol) was added dropwise. The exothermic reaction was cooled with an ice-water bath. The reaction mixture was stirred at room temperature for 1 hour. The product mixture was diluted with methylene chloride and washed with water. The organic extract was dried over anhydrous sodium sulfate, filtered, and concentrated under vacuum. This intermediate mixture of mono- and bis-mesylates was used in the following cyclization reaction without further purification.

A mixture of the above mesylates (13.1 g) and cesium carbonate (13.1 g, 40 mmol) in anhydrous DMF (600 mL) was heated at 105° C. for 6 hours with vigorous stirring. The reaction mixture was filtered through a pad of Celite and the solid filtered washed with DMF. The filtrates were combined and concentrated under vacuum. The residue was partitioned between ethyl acetate and brine. The organic extract was washed with brine, dried over anhydrous sodium sulfate, filtered and concentrated under vacuum to provide the title compound as a mixture of two diastereoisomers. The mixture was used in the following step without any further purification.

¹H NMR (400 MHz, CDCl₃) δ 7.37 (dd, J=1.8, 6.8 Hz, 1H), 7.21 (m, 1H), 7.10 (t, J=8.6 Hz, 1H), 4.86-4.49 (m), 4.08 (s), 4.07 (s), 4.2-2.8 (m), 3.17 (s), 3.13 (s), 1.8-1.4 (br m), 1.25 (s), 1.10 (s), 0.95 (s), 0.91 (s). ES-MS M+H=576 for both isomers.

### Step 15: (4R)-11-(3-Chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione

To a cold (0° C.) solution of (4R)-11-(3-chloro-4-fluorobenzyl)-9-methoxy-2,5,5-trimethyl-4-(tetrahydro-2H-pyran-2-yloxy)-3,4,5,6,12,13-hexahydro-2H[1,4]diazocino[2,1-a]2,6-naphthyridine-1,8,10(11H)-trione (3.3 g, 5.7 mmol) in anhydrous methylene chloride (35 mL), solution of boron tribromide in methylene chloride (22.9 mL, 1.0 M, 22.9 mmol) was added. The reaction mixture was stirred at room temperature for 2 hours. Reaction mixture was cooled with an ice-water bath, quenched with water (20 mL), and stirred at room temperature for 30 minutes. The product mixture was diluted with methylene chloride (100 mL) and water (50 mL). Small amount of methanol was added to dissolve the gummy material in the organic phase. The aqueous phase was separated and extracted with methylene chloride. The organic extracts were combined and washed with brine, dried over anhydrous magnesium sulfate, filtered, and concentrated under vacuum. The residue was purified with preparative HPLC on a 50×250 mm Xterra 10 micron column eluted with a 20-35% acetonitrile-water gradient at 100 mL/minute over 50 minutes. Fractions of the faster eluting major isomer were collected and lyophilized to afford the major isomer as white solid.

Isomer A-1: ¹H NMR (500 MHz, CDCl₃) δ 13.1 (br s, 1H), 7.35 (dd, J=2.2, 6.8 Hz, 1H), 7.20 (m, 1H), 7.13 (t, J=8.8 Hz, 1H), 4.84 (d, J=14.6 Hz, 1H), 4.74 (d, J=14.6 Hz, 1H), 4.59 (d, J=14.6 Hz, 1H), 3.73 (dd, J=14.9, 9.5 Hz, 1H), 3.43 (m, 3H), 3.18 (s, 3H), 3.13 (d, J=14.6 Hz, 1H), 3.01 (d, J=14.9 Hz, 1H), 2.92 (m, 1H), 2.52 (dt, J=15.6, 4.9 Hz, 1H), 1.21 (s, 3H), 0.94 (s, 3H). (ES MS M+1=478.1).

Fractions of the slower eluting minor isomer were collected and lyophilized. The solid was further purified with preparative HPLC on a 50×250 mm Xterra 10 micron column eluted with a 20-37% acetonitrile-water gradient at 100 mL/minute over 50 minutes. Collection and lyophilization of appropriate fractions provided the minor isomer as pale yellow solid.

Isomer B-1: ¹H NMR (500 MHz, CDCl₃) δ 13.0 (br s, 1H), 7.36 (dd, J=2.0, 6.8 Hz, 1H), 7.20 (m, 1H), 7.13 (t, J=8.6 Hz, 1H), 4.76 (d, J=14.6 Hz, 1H), 4.60 (d, J=10.0 Hz, 1H), 4.57 (d, J=10.0 Hz, 1H), 3.74 (d, J=14.9 Hz, 1H), 3.61 (d, J=14.4 Hz, 1H), 3.45-3.35 (m, 4H), 3.25 (s, 3H), 2.92 (m, 1H), 2.48 (dt, J=15.8, 4.6 Hz, 1H), 1.15 (s, 3H), 0.87 (s, 3H). (ES MS M+1=478.2).

From NMR studies conducted with pure samples of the individual isomers A and B dissolved in CD₂Cl₂ for over 24 hours, it was determined that Isomers A and B are related as diastereomers due to the presence of the chiral (R) hydroxy group in the 8-membered ring and the different orientations (i.e., atropisomerism) of the amide group in the 8-membered ring as a result of the restricted rotation of this amide group relative to the bicyclic core. No equilibration between the two isomers was observed in the NMR studies. Adopting the Helical nomenclature for assigning atropisomers (see Prelog et al., Angew. Chem. Int. Ed. Engl. 1992, 21: 567-583) the appropriate name for:

Isomer A is M-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione; and for:

Isomer B is P-(4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione.

### Example 2

### (4S)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione

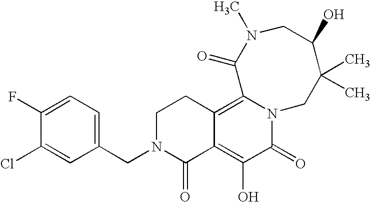

The title compound was prepared in accordance with the procedure set forth in Example 1 using L(+)-pantolactone in place of D(−)-pantolactone. ES MS M+1=478.2.

The title 4S isomer can be further resolved into two isomers A-2 and B-2 in a manner analogous to that described in Example 1.

### Example 3

### Stereoisomers of 11-(3-Chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione

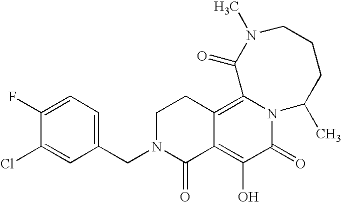

### Step 1: 5-(Methylamino)pentan-2-ol

A mixture of γ-valerolactone (5.0 g, 49.9 mmol) and methylamine (75 mL, 2 M in methanol) in methanol (50 mL) was stirred at room temperature overnight. The product mixture was concentrated under vacuum. This intermediate methylamide was concentrated from benzene to remove residual methanol and was used in the following step without further purification. To a cold (0° C.) solution of the above amide (2.0 g, 15.3 mmol) in anhydrous THF, a solution of lithium aluminum hydride (15.2 mL, 2M) in THF was added. The reaction mixture was stirred at room temperature for 30 minutes, and heated at 65° C. overnight. The product mixture was cooled to 0° C. and treated successively with water (1.2 mL), 15% aq sodium hydroxide (1.2 mL), and water (3.6 mL). The resultant suspension was diluted with ether, and filtered with a pad of Celite. The solid filtered was washed with methylene chloride. The organic filtrates were combined and concentrated under vacuum to provide the title compound.

### Step 2: 11-(3-Chloro-4-fluorobenzyl)-9-methoxy-2,6-dimethyl-3,4,5,6,12,13-hexahydro-2H-[1,4]diazocino[2,1-a]-2,6-naphthyridine-1,8,10(11H)-trione

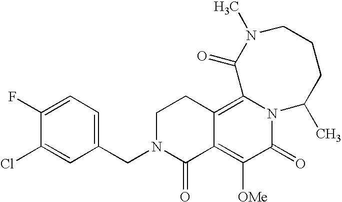

The title compound was prepared in a manner similar to that described in Example 1, steps 13 to 14, substituting (3R)-2,3-dimethyl-4-(methylamino)-3-(tetrahydro-2H-pyran-2-yloxy)-butanol with 5-(methylamino)pentan-2-ol in step 13. The product was a mixture of four diastereomers, being atropisomeric at the amide moiety of the eight-membered ring lactam and enantiomeric at the 6-methyl position. They were separated via supercritical-fluid chromatography (SCF) over a ChiralPak AD, 10 micron, 2×25 cm column with 90% carbon dioxide/10% methanol as the eluent.

### Step 3: 11-(3-Chloro-4-fluorobenzyl)-9-hydroxy-2,6-dimethyl-3,4,5,6,12,13-hexahydro-2H-[1,4]diazocino[2,1-a]-2,6-naphthyridine-1,8,10(11H)-trione

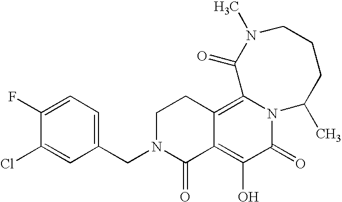

Each of the four diastereomers from step 2 was independently deprotected by stirring in a solution in 30% HBr in acetic acid for at room temperature for 1 hour and then stripping the reaction mixture to dryness. The products mixture was purified by reverse phase HPLC over a Phenomenex Synergi Polar-RP 80A, 4 micron, 100×21.2 mm column using a 70:30, 0.1% TFA in water/acetonitrile to 60:40, 0.1% TFA in water/acetonitrile gradient over 30 minutes.

### Diastereomer A-3:

¹H NMR (400 MHz, CDCl₃) δ 7.35 (d, J=6.6 Hz, 1H), 7.18 (br signal, 1H), 7.13 (t, J=8.4 Hz, 1H), 4.77 (d, J=14.7 Hz, 1H), 4.55 (d, J=14.7 Hz, 1H), 4.05-4.09 (m, 1H), 3.46-3.53 (m, 1H), 3.32-3.42 (m, 2H), 3.14 (s, 3H), 3.01-3.08 (m, 1H), 2.55-2.72 (m, 2H), 1.86 (br signal), 1.72 (d, J=6.8 Hz, 3H). (ES MS exact mass M+1=448.1428)

### Diastereomer B-3:

¹H NMR (400 MHz, CDCl₃) δ 7.35 (dd, J=6.8, 2.1 Hz, 1H), 7.18 (br signal, 1H), 7.12 (t, J=8.6 Hz, 1H), 4.76 (d, J=14.8 Hz, 1H), 4.56 (d, J=14.8 Hz, 1H), 4.01-4.03 (m, 1H), 3.46-3.69 (m, 1H), 3.33-3.40 (m, 2H), 3.08 (s, 3H), 3.11-3.16 (m, 1H), 3.01-3.07 (m, 1H), 2.52-2.74 (m, 2H), 1.85 (br signal), 1.70 (d, J=6.8 Hz, 3H). (ES MS exact mass M+1=448.1427)

### Diastereomer C-3:

¹H NMR (400 MHz, CDCl₃) δ 7.35 (dd, J=7.0, 2.0 Hz, 1H), 7.18 (br signal, 1H), 7.12 (t, J=8.6 Hz, 1H), 5.71 (br signal, 1H), 4.77 (d, J=14.8 Hz, 1H), 4.55 (d, J=14.8 Hz, 1H), 3.45-3.51 (m, 1H), 3.27-3.40 (m, 2H), 3.12-3.17 (m, 1H), 3.06 (s, 3H), 2.93-3.00 (m, 1H), 2.50-2.57 (m, 1H), 1.99-2.01 (m, 2H), 1.72-1.79 (m, 2H), 1.33 (d, J=7.1 Hz, 3H). (ES MS exact mass M+1=448.1428)

### Diastereomer D-3:

¹H NMR (400 MHz, CDCl₃) δ 7.35 (dd, J=6.8, 1.8 Hz, 1H), 7.19 (br signal, 1H), 7.12 (t, J=8.5 Hz, 1H), 5.70 (br signal, 1H), 4.77 (d, J=14.6 Hz, 1H), 4.55 (d, J=14.8 Hz, 1H), 3.45-3.51 (m, 1H), 3.287-3.40 (m, 2H), 3.11-3.15 (m, 1H), 3.05 (s, 3H), 2.92-3.00 (m, 1H), 2.49-2.57 (m, 1H), 1.96-2.01 (m, 2H), 1.73-1.77 (m, 2H), 1.33 (d, J=7.1 Hz, 3H). (ES MS exact mass M+1=448.1457)

### Example 4

### Oral Compositions

As a specific embodiment of an oral composition of a compound of this invention, 50 mg of Isomer A-1 of Example 1 is formulated with sufficient finely divided lactose to provide a total amount of 580 to 590 mg to fill a size 0 hard gelatin capsule. Encapsulated oral compositions containing Isomer B-1 of Example 1, an isolated atropisomer of the compound of Example 2, or any one of Diastereomers A-3 to D-3 of Example 3 can be similarly prepared.

### Example 5

### HIV Integrase Assay

### Strand Transfer Catalyzed by Recombinant Integrase

Assays for the strand transfer activity of integrase were conducted in accordance with WO 02/30930 for recombinant integrase. Representative compounds of the present invention exhibit inhibition of strand transfer activity in this assay. For example, the compounds prepared in Examples 1 to 3 were tested in the integrase assay and found to have the following IC₅₀ values:

| Compound | IC₅₀ (μM) |
|---|---|
| Ex. 1 - Isomer A-1 | 0.014 |
| Ex. 1 - Isomer B-1 | 0.021 |
| Ex. 2 | 0.003 |
| Ex. 3 - Diasteromer | 0.017 |
| A-3 | |
| Ex. 3 - Diasteromer | 0.017 |
| B-3 | |
| Ex. 3 - Diasteromer | 0.008 |
| C-3 | |
| Ex. 3 - Diasteromer | 0.008 |
| D-3 | |

Further description on conducting the assay using preassembled complexes is found in Wolfe, A. L. et al., J. Virol. 1996, 70: 1424-1432, Hazuda et al., J. Virol. 1997, 71: 7005-7011; Hazuda et al., Drug Design and Discovery 1997, 15: 17-24; and Hazuda et al., Science 2000, 287: 646-650.

### Example 6

### Assay for Inhibition of HIV Replication

Assays for the inhibition of acute HIV infection of T-lymphoid cells were conducted in accordance with Vacca, J. P. et al., Proc. Natl. Acad. Sci. USA 1994, 91: 4096. Representative compounds of the present invention exhibit inhibition of HIV replication in this assay (also referred to herein as the “spread assay”). For example, the compounds of Examples 1 to 3 were tested in this assay and found to have the following IC₉₅ values:

| | IC₉₅ (μM) |
|---|---|
| Compound | in the presence of 10% FBS |
| Ex. 1 - Isomer A-1 | 0.015 |
| Ex. 1 - Isomer B-1 | 0.031 |
| Ex. 2 | 0.031 |
| Ex. 3 - Diasteromer | 0.125 |
| A-3 | |
| Ex. 3 - Diasteromer | 0.011 |
| B-3 | |
| Ex. 3 - Diasteromer | 0.250 |
| C-3 | |
| Ex. 3 - Diasteromer | 0.008 |
| D-3 | |

### Example 7

### Assay for Inhibition of HIV Integrase Mutant Virus Replication

An assay for measuring the inhibition of acute HIV infection with HeLa P4-2 cells in a single cycle infectivity assay was conducted using methods described in Joyce et al., J. Biol. Chem. 2002, 277: 45811, Hazuda et al., Science 2000, 287: 646, and Kimpton et al, J. Virol. 1992, 66: 2232. Proviral plasmids encoding viruses containing specific mutations in the integrase gene (T66I/S153Y, N155S, or F121Y) were generated by site-directed mutagenesis, and viruses were produced by transfecting 293T cells with the appropriate proviral plasmids. Representative compounds of the present invention exhibit inhibition of HIV replication in the mutant assays. For example, the compounds of Examples 1 to 3 were found to have the following IC₅₀ values in these assays:

| | Number of fold shift in | | |
|---|---|---|---|
| | IC50 versus wild type IIIB¹ | | |
| Compound | T66I/S153Y | N155S | F121Y |
| Ex. 1 - Isomer A-1 | 1 | 5 | 2 |
| Ex. 1 - Isomer B-1 | 21 | 46 | 7 |
| Ex. 2 | 3 | 32 | 9 |
| Ex. 3 - Diastereomer | 2 | 26 | 8 |
| A-3 | | | |
| Ex. 3 - Diastereomer | 1 | 1 | 1 |
| B-3 | | | |
| Ex. 3 - Diastereomer | 4 | 8 | 3 |
| C-3 | | | |
| Ex. 3 - Diastereomer | 5 | 24 | 5 |
| D-3 | | | |

¹A number “x” in the table where x > 1 means the compound is x-fold less potent against the mutant compared to its potency against the wild type.

### Example 8

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention that were tested in the spread assay (see Example 6) were examined for cytotoxicity up to a concentration of 10 micromolar, and no cytotoxicity was exhibited. In particular, the compounds set forth in Examples 1 to 3 exhibited no cytotoxicity at concentrations up to 10 micromolar.

While the foregoing specification teaches the principles of the present invention, with examples provided for the purpose of illustration, the practice of the invention encompasses all of the usual variations, adaptations and/or modifications that come within the scope of the following claims.

1. An individual stereoisomer of a compound of Formula I having two sources of chirality in the 8-membered ring, or a pharmaceutically acceptable salt thereof: 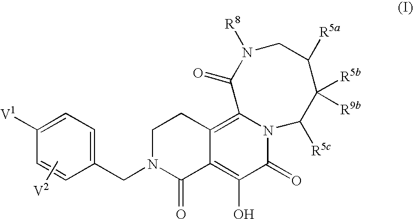
wherein:
R^{5a} is H or OH;
R^{5b} and R^{9b} are either both H or both CH₃;
R^{5c} is H or CH₃;
R⁸ is C₁₋₃ alkyl; and
V¹ and V² are each independently Br, Cl, F, or I;
and provided that
(A) when R^{5b} and R^{9b} are both H, then R^{5a} is H and R^{5c} is CH₃; and
(B) when R^{5b} and R^{9b} are both CH₃, then R^{5a} is OH and R^{5c} is H.

2. The individual stereoisomer according to claim 1, or a pharmaceutically acceptable salt thereof, wherein R⁸ is CH₃.

3. The individual stereoisomer according to claim 1, or a pharmaceutically acceptable salt thereof, wherein V¹ is F; and V² is Br, Cl, or F.

4. The individual stereoisomer according to claim 1, or a pharmaceutically acceptable salt thereof, wherein V¹ is F; and V² is Cl.

5. The individual stereoisomer according to claim 1, or a pharmaceutically acceptable salt thereof, wherein V¹ is F; and V² is Cl in the meta position of the benzyl moiety.

6. The individual stereoisomer according to claim 1, or a pharmaceutically acceptable salt thereof, wherein one of the sources of chirality is atropisomerism.

7. The individual stereoisomer according to claim 1, which is selected from the group consisting of:
Isomer A-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione;
Isomer B-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione;
Isomer A-2 of (4S)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione;
Isomer B-2 of (4S)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione;
Diastereomer A-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione;
Diastereomer B-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione;
Diastereomer C-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione;
Diastereomer D-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione;
and pharmaceutically acceptable salts thereof.

8. The individual stereoisomer according to claim 7, which is selected from the group consisting of:
Isomer A-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione;
Diastereomer B-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione;
and pharmaceutically acceptable salts thereof.

9. The individual stereoisomer according to claim 8, which is Isomer A-1 of (4R)-11-(3-chloro-4-fluorobenzyl)-4,9-dihydroxy-2,5,5-trimethyl-3,4,5,6,12,13-hexahydro-2H[1,4]diazocino[2,1-a]-2,6-naphthyridine-1,8,10(11H)-trione, or a pharmaceutically acceptable salt thereof.

10. The individual stereoisomer according to claim 8, which is Diastereomer B-3 of 11-(3-chloro-4-fluorobenzyl)-9-hydroxy-2,6-dimethyl-3,4,5,6,12,13-hexahydro-2H[1,4]diazocino[2,1-a]-2,6-naphthyridine-1,8,10(11H)-trione, or a pharmaceutically acceptable salt thereof.

11. A pharmaceutical composition comprising an effective amount of a stereoisomer according to claim 1, or a pharmaceutically acceptable salt thereof, and a pharmaceutically acceptable carrier.

12. A method for the inhibition of HIV integrase in a subject in need thereof which comprises administering to the subject an effective amount of the stereoisomer according to claim 1, or a pharmaceutically acceptable salt thereof.

13. A method for the treatment or prophylaxis of infection by HIV or for the treatment, prophylaxis, or delay in the onset or progression of AIDS in a subject in need thereof, which comprises administering to the subject an effective amount of the stereoisomer according to claim 1, or a pharmaceutically acceptable salt thereof.

14. (canceled)

15. A process for preparing a compound of Formula II: 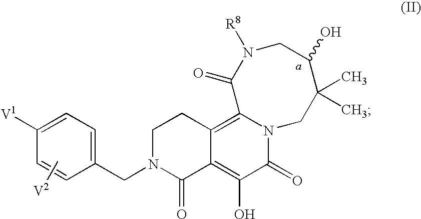
which comprises:
(A) treating a compound of Formula III: 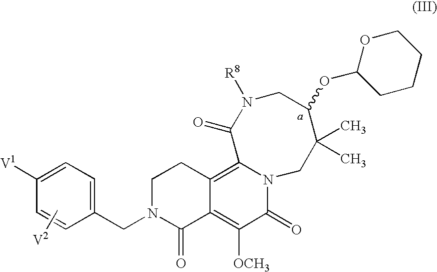
with acid to obtain Compound II; wherein stereocenter “a” is in the R or the S configuration; R⁸ is C₁₋₃ alkyl; and V¹ and V² are each independently Br, Cl, F, or I.

16. The process according to claim 15, which further comprises:
(B) sequentially treating a compound of Formula IV: 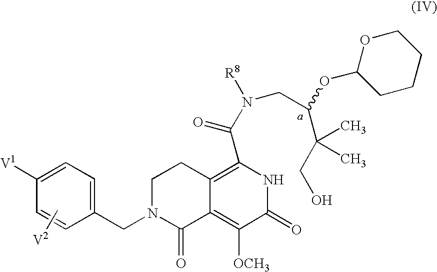
first with a sulfonic anhydride or a sulfonyl halide in the presence of a first base and then with a second base to obtain Compound III.

17. The process according to claim 15, wherein Compound II is a compound of Formula II-A: 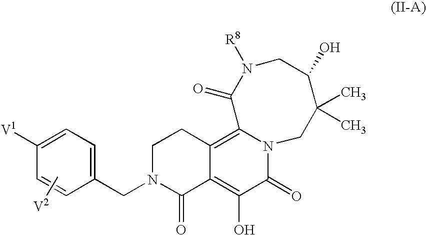
and Compound III is a compound of Formula III-A:

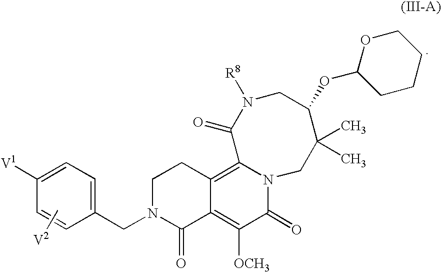

18. The process according to claim 17, which further comprises:
(B) sequentially treating a compound of Formula IV-A: 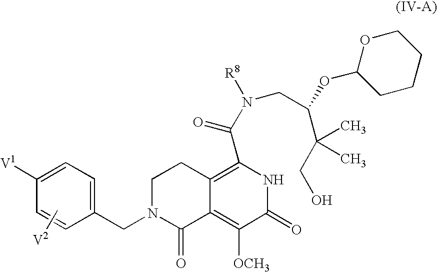
first with a sulfonic anhydride or a sulfonyl halide in the presence of a first base and then with a second base to obtain Compound III-A.

19. The process according to claim 15, wherein R⁸ is CH₃; V¹ is F; and V² is Cl in the meta position of the benzyl moiety.

20. A compound which is a compound of Formula III: 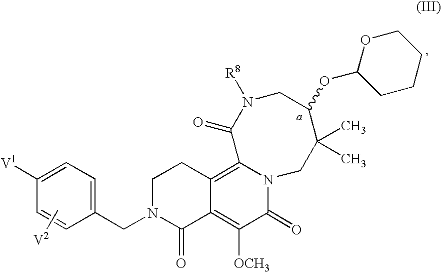
a compound of Formula IV: 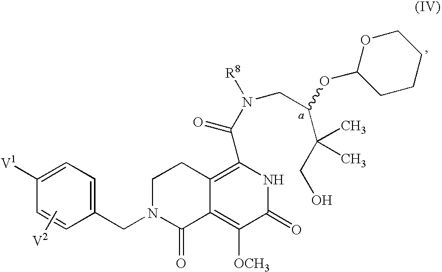
or a salt thereof; wherein stereocenter “a” is in the R or the S configuration; R⁸ is C₁₋₃ alkyl; and V¹ and V² are each independently Br, Cl, F, or I.
